(19) United States
(12) Patent Application Publication
(10) Pub. No.: US 2026/0274775 A1
(43) Pub. Date: Sep. 17, 2026

(54) METHOD FOR FORMING METHANOL AND FORMIC ACID

(71) Applicant: QUEEN'S UNIVERSITY OF BELFAST, Belfast (GB)

(72) Inventors: Haresh MANYAR, Belfast (GB); Dipti WAGH, Belfast (GB)

(73) Assignee: QUEEN'S UNIVERSITY OF BELFAST, Belfast (GB)

(21) Appl. No.: 19/472,061

(22) PCT Filed: Apr. 3, 2024
(86) PCT No.: PCT/EP2024/059022

§ 371 Date: Oct. 2, 2025

(30) Foreign Application Priority Data
Apr. 5, 2023 (GB) 2305083.4

(51) Int. Cl. C07C 29/159; C07C 31/04; C07C 51/00; C07C 53/02

(52) U.S. Cl. CPC C07C 29/159; C07C 31/04; C07C 51/00; C07C 53/02

(57) ABSTRACT

A method of forming methanol and/or formic acid, comprising irradiating a mixture comprising water, carbon dioxide, carbon absorbent, and a photocatalyst, with visible light and UV light to reduce the carbon dioxide and form methanol and/or formic acid. The photocatalyst comprises zinc oxide and manganese oxide.

### FIELD OF THE INVENTION

The present invention relates to a method of forming methanol, formic acid, or a mixture of methanol and formic acid, apparatus therefor, and use of a photocatalyst in the reduction of carbon dioxide.

### BACKGROUND

At present, there is an ongoing global trend observed in relation to increases in oil and gas prices. This increase has further enhanced the instability of the current energy system which is heavily reliant on the use of fossil fuels, consequently signifying the need for a sustainable renewable energy supply.

Capturing carbon dioxide (CO₂) from the atmosphere and utilising it to produce hydrocarbon fuels could help with this issue. Carbon capture and utilisation (CCU) can assist in overcoming the global energy crisis, providing energy security while combatting climate change simultaneously.

Various methods of converting carbon dioxide to methanol fuels using photocatalytic reduction are known. An object of the present invention is to provide an improved method of forming methanol and/or formic acid from carbon dioxide that has greater viability for scale up to a commercial scale.

### SUMMARY

According to one aspect of the present invention, there is provided a method of forming methanol, comprising:
irradiating a mixture comprising water, carbon dioxide, carbon absorbent, and a photocatalyst, with visible light and UV light to reduce the carbon dioxide and form methanol,
wherein the photocatalyst comprises zinc oxide and manganese oxide.

According to a second aspect, there is provided a method of forming formic acid, comprising:
irradiating a mixture comprising water, carbon dioxide, carbon absorbent, and a photocatalyst, with visible light and UV light to reduce the carbon dioxide and form formic acid,
wherein the photocatalyst comprises zinc oxide and manganese oxide.

According to a third aspect, there is provided a method of forming methanol and formic acid, comprising:
irradiating a mixture comprising water, carbon dioxide, carbon absorbent, and a photocatalyst, with visible light and UV light to reduce the carbon dioxide and form methanol and formic acid,
wherein the photocatalyst comprises zinc oxide and manganese oxide.

Optionally, the method carried out at ambient temperature, or at ambient pressure, or both ambient temperature and ambient pressure. That is, no additional heat energy is required, and no increased pressure regime is required. The method of the present invention can be carried out in the absence of additional heat energy and any additional pressure.

Manganese oxide can have a number of forms. These include but are not limited to MnO, MnO2, Mn2O3, and Mn3O4, with various crystal forms including octahedral, tetrahedral closed cubic packed etc, known in the art. All such forms are useable with the present invention.

Optionally, the manganese oxide has an octahedral framework, such as cryptomelane, such as OMS-2.

Where the present invention uses zinc oxide and manganese oxide, the present invention can be based on the direct Z-scheme mechanism as discussed hereinafter.

The present invention includes the additional of one or more additional metals and/or compounds to the photocatalyst. Such additional metals and/or compounds include copper. As such, the present invention can be based on the indirect Z-scheme mechanism as discussed hereinafter.

Where the present invention uses zinc oxide and manganese oxide, the present invention can be based on the direct Z-scheme mechanism as discussed hereinafter.

The carbon absorbent is an agent to enhance the amount of carbon dioxide absorption in water, or together with the carbon absorbent. It is known that various compounds can increase the capture of carbon dioxide alongside water. Such absorption is typically intended to achieve a ‘saturated’ solution for maximum processing, but the invention is not limited thereto.

Optionally, the carbon absorbent is at least one of an amine or oxide. Various amines and oxides are known in the art for enhancing the amount of carbon dioxide absorption in water. This is sometimes referred to as ‘carbon capture’.

Optionally, the carbon absorbent comprises a mixture or more than one compound.

Optionally, the carbon absorbent comprises a mixture or more than one liquid compound.

Optionally, the carbon absorbent is selected from the group comprising: trimethylamine (TEA), dimethylformamide (DMF), calcium oxide, magnesium oxide and any mixture thereof.

The water and carbon absorbent can form a mixture in advance of carbon dioxide addition, and can form a mixture able to be recycled for reuse in the method of the present invention.

Optionally, the volume ratio of the carbon absorbent to water is greater than 1:1.

Optionally, the volume ratio of the carbon absorbent to water is greater than 3:1.

Optionally, the carbon absorbent is a mixture of trimethylamine and dimethylformamide, and wherein the volume ratio of the carbon absorbent to water is 4:1.

Optionally, where the carbon absorbent comprises a mixture or more than one compound, the ratio of such compounds is variable to maximise the absorption of carbon dioxide by the carbon absorbent and water mixture.

Optionally, the weight ratio of zinc oxide to manganese oxide in the photocatalyst can be any suitable ratio, such as in the range 50:1 to 1:50.

Optionally, the weight ratio of zinc oxide to manganese oxide in the photocatalyst is in the range 10:1 to 1:10, such as 5:1 to 1:5, including for example 4:1, 3:2, 2:3 and 1:4.

Optionally, the weight ratio of zinc oxide to manganese oxide in the photocatalyst is in the range 3:1 to 5:1, such as 4:1.

The term ‘ratio’ as used herein is not limited to the exact numerals used to define any ratio, but may include any reasonable variation in numeral scope.

In the method of the present invention, the photocatalyst is simultaneously irradiated with visible light and UV light.

Optionally, the UV light includes a wavelength in the range of 350-400 nm.

Optionally, the visible light includes a wavelength in the range of 600 nm to 800 nm.

In the method of the present invention, the photocatalyst may be irradiated with visible light and UV light using any suitable light sources or sources. Without limitation to the invention.

Optionally, the irradiation is provided by direct sunlight.

Optionally, the method of the present invention further comprises the steps of:
passing the carbon dioxide through the water and carbon absorbent to provide a wholly or substantially saturated solution; and
passing the saturated solution across the photocatalyst.

The carbon dioxide may be passed through the water and carbon absorbent in any suitable process or arrangement to achieve the carbon capture. As carbon dioxide is a gas at room temperature and pressure, one convenient method is to pass carbon dioxide gas through a liquid mixture of water and carbon absorbent. Suitable apparatus for same involving gaseous spargers or distributors are well known in the art. A pump can be used to pump the carbon dioxide through the water and carbon absorbent, until a suitable degree of absorption has occurred, optionally until a ‘saturated solution’ is achieved.

A wholly or substantially saturated solution can then be passed across the photocatalyst in any suitable manner. Optionally, the photocatalyst is on a suitable support or coated as film or has a suitable packed structure, to allow free passing of the solution thereacross and therethrough, to achieve the photocatalytic process of converting carbon dioxide into methanol, formic acid, or a mixture thereof. The chemical pathway for such reaction is well known in the art.

The photocatalyst could be on a fixed bed.

Optionally, the photocatalyst is within a suitable station or housing allowing the passage of irradiated light therethrough, and the passage of the solution is confined or restrained to facilitate the formation of methanol, formic acid, or a mixture thereof.

Optionally, the method of the present invention further comprises the step of:
separating the methanol from the water and the carbon absorbent.

The separation may be achieved by any suitable process or apparatus. One example is a gas/liquid separator known in the art, able to separator the desired products from the water and carbon absorbent.

Optionally, the method of the present invention further comprises the step of:
recycling the water and carbon absorbent for further carbon dioxide absorption.

According to a further aspect of the present invention, there is provided apparatus for forming methanol, formic acid, or a mixture of methanol and formic acid, comprising:
a mixing station for mixing water, carbon dioxide, and carbon absorbent,
a photocatalyst station configured to be irradiated with visible light and UV, wherein the photocatalyst comprises zinc oxide and manganese oxide.

The mixing station may be any suitable area or vessel or container, able to allow the absorption of carbon dioxide by the water and carbon absorbent.

Optionally, the mixing station comprises a carbon dioxide gaseous streamer able to stream carbon dioxide through a mixture of water and carbon absorbent.

Optionally, the apparatus further comprises a gas/liquid separator after the photocatalyst station for separating the methanol, formic acid, or a mixture of methanol and formic acid, from the water and carbon absorbent.

Optionally, the apparatus further comprises a recycling pathway to pass the water and carbon absorbent after the photocatalyst station and any gas/liquid separator, to the mixing station.

Optionally, the apparatus further comprises one or more LEDs to provide the visible light and the UV light.

According to a further aspect of the present invention, there is provided use of a photocatalyst comprising zinc oxide and manganese oxide for the reduction of carbon dioxide and water into methanol, formic acid, or a mixture of methanol and formic acid.

Optionally, the use involves irradiation by visible light and UV light.

Optionally, the use involves a method as defined herein or apparatus as defined herein.

### BRIEF DESCRIPTION OF THE DRAWINGS

The drawings show various embodiments of the present invention.

FIGS. 1a and 1b are Z-Scheme photocatalytic systems for CO₂ reduction. 1a, Indirect Z-Scheme Mechanism. 1b, Direct Z-Scheme Mechanism

FIGS. 2a and 2b are photocatalytic reduction of CO2 by various catalysts (ZnO, OMS-2 and ZnO/OMS-2 (4:1)). 2a, Methanol yield. 2b, Formic acid yield. Reaction condition: DMF/TEA/H2O (12/4/4), 50 mg catalyst loading, UV (365 nm) and visible light.

FIGS. 3a and 3b are photocatalytic reduction of CO2 By various catalysts (ZnO, Cu/OMS-2 and ZnO/Cu/OMS-2 (4:1)). 3a, Methanol yield. 3b, Formic acid yield. Reaction condition: DMF/TEA/H2O (12/4/4), 50 mg catalyst loading, UV (365 nm) and visible light.

FIGS. 4a and 4b are effects of catalyst composition on 4a. Methanol formation and 4b. Formic Acid formation in the photocatalytic reaction. Reaction condition: DMF/TEA/H2O (12/4/4), 50 mg catalyst loading, UV (365 nm) and visible light.

FIGS. 5 and 5b are photocatalytic reduction of CO2 under UV, visible and UV-visible irradiation by ZnO, OMS-2 and ZnO/OMS-2 (4:1) catalyst. 5a, Methanol yield. 5b, Formic acid yield. Reaction condition (Batch): DMF/TEA/H2O (12/4/4), 50 mg catalyst loading.

FIGS. 6a and 6b are effects of Water, DMF and TEA in photocatalytic CO₂ reduction. 6a, Methanol yield. 6b, Formic acid yield. Reaction condition (Batch): 50 mg catalyst loading, under UV (365 nm) and visible irradiation.

FIG. 7 is a schematic view of a continuous flow photocatalytic reduction of CO₂ to methanol.

### DETAILED DESCRIPTION

The present invention focuses on methods, photocatalysts and photoreactor designs to enhance the efficiency of the photocatalytic reduction of CO₂. This is particularly but not exclusively centred around a UV-visible activated direct Z-scheme photocatalyst that takes advantage of the fact that approximately 43% of sunlight is comprised of the visible component of the spectrum. Hence, the potential for scale-up is much greater through the use of visible light. In addition, the application of visible light is less energy intensive compared with that of UV irradiation.

LEDs are considered the ideal light source for photocatalytic applications as their use provides huge benefits in regards to selecting an appropriate light wavelength to match the characteristics of the photocatalyst material.

The present invention particularly covers a method of forming methanol and formic acid, comprising:
irradiating a mixture comprising water, carbon dioxide, carbon absorbent, and a photocatalyst, with visible light and UV light to reduce the carbon dioxide and form methanol and formic acid,
wherein the photocatalyst comprises zinc oxide and manganese oxide.

The present invention also particularly covers apparatus for forming methanol, formic acid, or a mixture of methanol and formic acid, comprising:
a mixing station for mixing water, carbon dioxide, and carbon absorbent,
a photocatalyst station configured to be irradiated with visible light and UV, wherein the photocatalyst comprises zinc oxide and manganese oxide.

In this way, the present invention also covers the use of a photocatalyst comprising zinc oxide and manganese oxide for the reduction of carbon dioxide and water into methanol, formic acid, or a mixture of methanol and formic acid.

The synthesis of Zinc Oxide Nanoparticles is well known in the art. By way of example only, the preparation of ZnO nanoparticles can follow the sol-gel method. At first, Zn(CH₃COO)₂·2 H₂O (20 g) is mixed with deionised water and stirred for 20 minutes at 35° C. to produce a zinc acetate solution. NaOH powder (80 g) is mixed into deionised water and stirred for around 20 minutes at 35° C. for producing NaOH solution. After mixing both solutions, a titration reaction can be performed by the addition of ethanol drop-wise into the solution until the pH is in the range 9-13 to obtain a gel-like product.

The synthesis of Manganese Oxide is well known in the art. By way of example only, the preparation of Manganese Oxide (OMS-2) can also follow the sol-gel method. A solution of KMnO4 is added to maleic acid to form a dark brown sol at room temperature. The resultant water-gel product was decanted, and the resultant gel was transferred to a filter funnel, followed by heating in air. Finally, the resultant particles can be calcined in air. The final chemical composition is KMn₈O₁₆·nH₂O.

The direct Z-scheme photocatalyst, Zn/OMS-2, was prepared mechanochemically by grinding the individual catalyst materials together for 30 min. Photocatalysts can be characterised by different methods of analysis, such as X-ray Diffraction (XRD), Scanning Electron Microscopy (SEM), Fourier Transform Infrared Spectroscopy (FT-IR), Ultraviolet-Visible (UV-Vis) spectroscopy and Brunauer-Emmett-Teller (BET) surface area analysis. Surface area, total pore volume and average pore diameter can be measured by N₂ adsorption-desorption isotherms at 77 K using Micromeritics ASAP 2010. Catalyst pore size can be calculated on the adsorption branch of the isotherms using the Barrett-Joyner-Helenda (BJH) method and the surface area was calculated using the Brunauer-Emmett-Teller (BET) method.

Various semi-conductors are designed to absorb light radiations. The threshold wavelength must provide sufficient photon energy to overcome the band gap between the valence bands (VBs) and the conduction bands (CBs). Equation 1 below sets out the band gap energy equation, rearranged to give the minimum wavelength that is required to promote the photoelectrons from the VB to CB. The wavelength of the light radiation influences the yield of CO₂ photocatalytic reduction products.

$$Minimum Wavelength$$

$${λ}_{m i n} = 1240 * band - gap energy \qquad Equation 1$$

ZnO has a large bandgap, and it is known that ZnO best absorbs at the wavelength 365 nm, which corresponds to the UV region.

Suitable manganese oxides such as OMS-2 absorb best at the wavelength 600-800 nm, this corresponds to the visible region.

Therefore, radiating a mixed or combined ZnO/OMS-2 photocatalyst with both UV and visible light will ensure maximum absorbance of the photons.

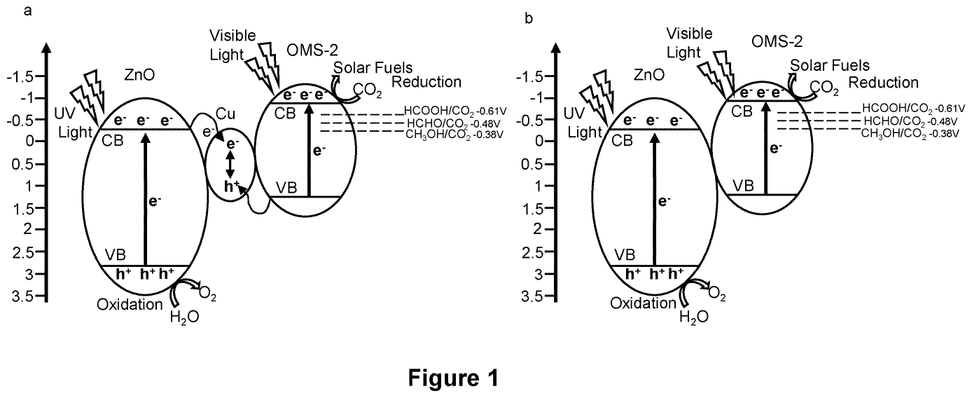

FIGS. 1a and 1b are Z-Scheme photocatalytic systems for CO₂ reduction. 1a, Indirect Z-Scheme Mechanism. 1b, Direct Z-Scheme Mechanism

As illustrated in FIG. 1, under the combined UV-visible light radiation, excited electrons are promoted from the valence band (VB) of ZnO to its CB. These electrons can rapidly recombine with holes in the VB, but with excitation from UV-visible light, both the ZnO and the OMS-2 (used by way of example) can both generate electron-hole pairs. The existence of an electric field thereinbetween now favours the recombination between the photogenerated electrons in the conduction band (CB) of OMS-2 and photogenerated holes in the VB of ZnO. So, the electrons in the ZnO CB and the holes from the OMS-2 VB recombine directly, leaving electrons in the OMS-2 CB and holes in the ZnO VB. As such, the holes in the VB of ZnO can react with water to produce, O₂ and H⁺ ions.

In this way, the CO₂ molecules are reduced to methanol, formic acid (and possibly formaldehyde) by the enriched electrons on the OMS-2 surface. A negative CB edge relative to the reduction potential of CO₂ will allow the transfer of electrons from the CB to CO₂, OMS-2 edge. Meanwhile, a positive VB edge relative to water potential will facilitate the transfer of holes from the VB to water, ZnO edge. At this point, the CO₂ is reduced to solar fuels, and water is oxidised to oxygen.

Various options and embodiments relating to the aspects covered by the present invention are described hereinabove, and the skilled reader can understand their application and uses.

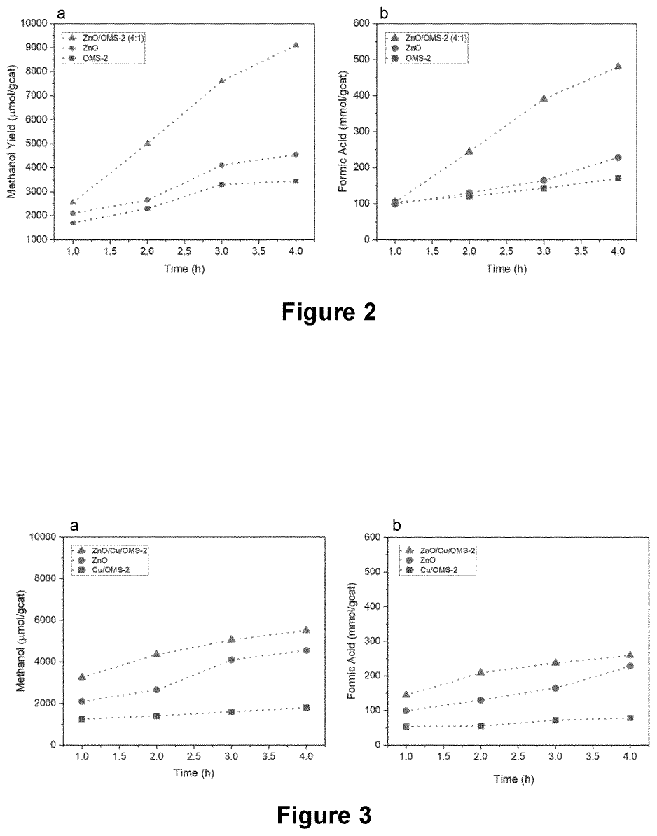

To confirm the outcome of the present invention, various examples of photocatalytic CO₂ reduction reactions were carried out using various comparative catalysts such as ZnO, OMS-2, Cu/OMS-2, against mixtures of ZnO/OMS-2 (FIGS. 2a,b) and ZnO/Cu/OMS-2 (FIGS. 3a,b).

The catalysts were prepared composed of different ratios of ZnO and OMS-2.

The examples for FIGS. 2 and 3 were carried out using 50 mg of each catalyst under UV-visible irradiation, and with a water and carbon absorbent mixture reaction medium comprised of 12 mL DMF, 4 mL deionised water and 4 mL TEA. The examples were carried out over 4 h reaction time with every 1 h sampling.

It can be observed in FIGS. 2a, b and 3a,b that that both methanol and formic acid respectively show a dramatic trend in which the use of a ZnO/OMS-2 (4:1) photocatalyst produced a higher yield. For both products, methanol and formic acid, Cu/OMS-2 resulted in the lowest yield, producing only 1800 μmol/gcat of methanol and 78 mmol/gcat formic acid. This is compared to the ZnO/OMS-2 photocatalyst where 9100 μmol/gcat of methanol and 480 mmol/gcat formic acid were obtained.

As shown in the results of FIGS. 2 and 3, ZnO/OMS-2 photocatalysts exhibit the highest yield of methanol and formic acid, followed by the ZnO/Cu/OMS-2 photocatalyst, then each of the ZnO, OMS-2 and lastly Cu/OMS-2 catalysts being lower yields. This concluded that Cu/OMS-2 and OMS-2 required ZnO to excite the electron-hole pairs to reduce CO2 and produce solar fuels.

This illustrate that irradiating the mixed metal oxide catalyst with UV-visible light produced the optimal yield of methanol and formic acid, 9100 μmol/gcat and 119 mmol/gcat respectively. From the results it is clear that for both methanol and formic acid production using visible light only produces a small yield therefore indicating that UV light is required to excite the photocatalyst. When UV only light was employed for the 4:1 mixed oxide photocatalyst, the yield was 5550 μmol/gcat for methanol and 226 mmol/gcat for formic acid. Using mixed light expresses an increase in 64% for methanol and 90% for formic acid yield.

From the graphs of FIGS. 2 and 3, it is evident that utilising mixed light produces a more substantial yield of products. Indeed, the present invention can provide a method of forming methanol that continuously provides at least more than 1500 or 2000 μmol/gcat/h, and a method of forming formic acid that continuously provides at least more than 100 or 150 mmol/gcat/h.

The mass and ratio of compounds in the photocatalyst can also effect the photocatalytic activity for the photocatalytic reduction of CO₂. Testing was carried out on the mass of photocatalyst being varied from 2 mg to 50 mg. Use of 2 mg with a photocatalyst mixture of ZnO/OMS-2 in a 4:1 ratio resulted in yields of 28750 μmol/gcat and 1201 mmol/gcat.

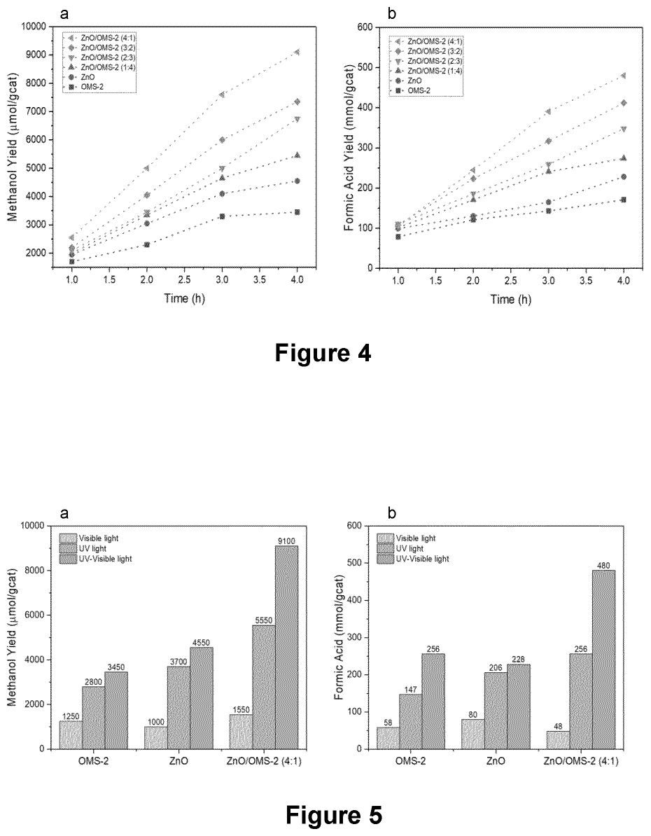

FIGS. 4a and 4b show the effects of variations in the catalyst composition ratio, on methanol formation in FIG. 4a, and in formic Acid formation in FIG. 4b (using a mixture of DMF/TEA/H2O (12/4/4), 50 mg catalyst loading, UV (365 nm) and visible light). FIG. 4a,b show various ZnO/OMS-2 ratios, all of which were superior to only using ZnO or OMS-2 as a photocatalyst with the same loading and under the same conditions.

FIGS. 5 and 5b show the differences in photocatalytic reduction of CO₂ under UV light only, visible light only, and by a combination of UV-visible irradiation, on the catalysts ZnO only, OMS-2 only, and a ZnO/OMS-2 (4:1) photo catalyst according to an embodiment of the present invention. FIG. 5a shows Methanol yields, and FIG. 5b shows Formic acid yields. The reaction medium was DMF/TEA/H2O (12/4/4), with 50 mg catalyst loading.

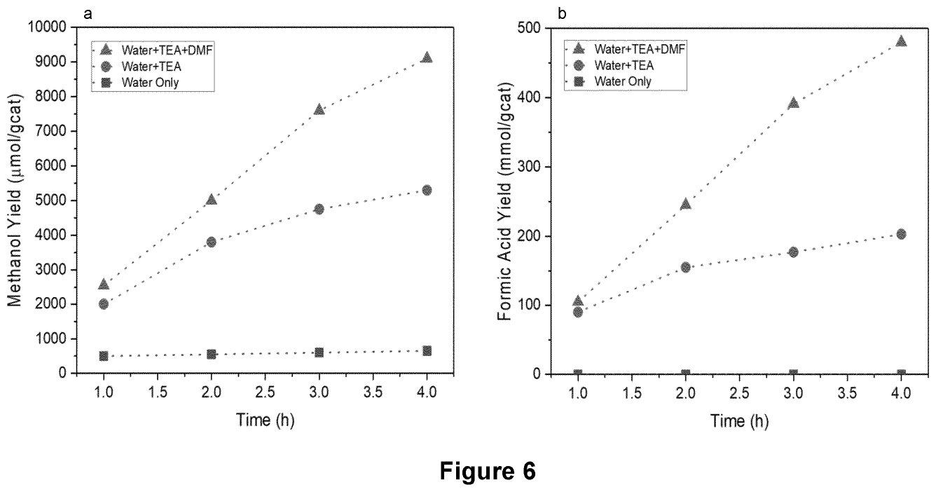

The effect of the carbon absorbent can be confirmed in FIGS. 6a and 6b. The carbon absorbent example of TEA has the role of a sacrificial donor, therefore increasing the amount of CO₂ that can be captured by water. DMF is an example of a carbon absorbent solvent with a higher CO₂ solubility than water. Therefore, increasing the amount of DMF in the reaction medium, increases the CO₂ solubility of the reaction and in turn a higher yield of products is formed. In addition to the large solubility, DMF has a smaller diffusion velocity due to its specific cation-solvating property, therefore stabilising the photoinduced charge separation between the photocatalyst and TEA.

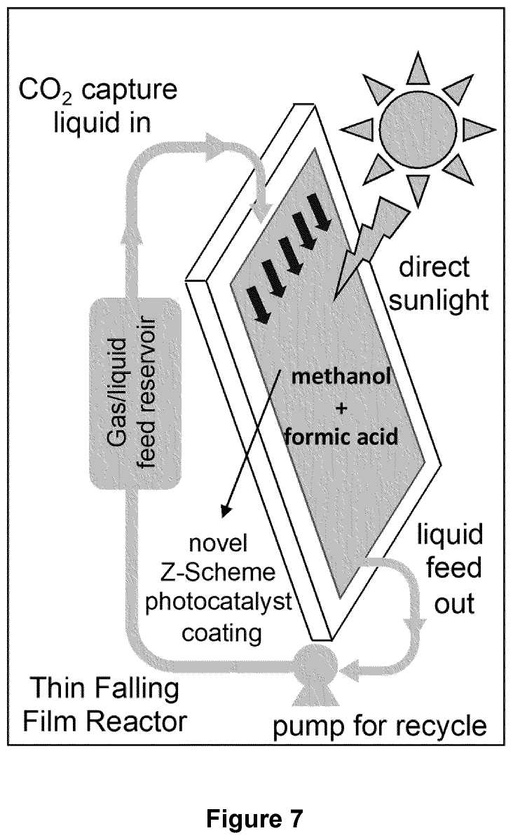

From the results in FIG. 6a,b, it is clear that a reaction medium based on more than just water, i.e. including a carbon absorbent is required to produce higher yield of products. FIG. 7b shows that no formic acid was produced when using water only, compared to 480 mmol/gcat of formic acid with both TEA and DMF, and 203 mmol/gcat with TEA only. FIG. 7a shows that methanol formed in 1 h was highest with TEA only, although over time TEA only slowed down the yield of methanol, indicating that another carbon absorbent such as DMF is also beneficial in the reaction medium to increase the solubility.

FIG. 7 shows an example of a continuous flow photoreactor. The reaction medium liquid can is pumped through the system using a HPLC pump, entering a gas-liquid T-junction in which it meets the gaseous CO₂ input stream. The liquid and gaseous streams are output from the T-junction in a segmented flow stream and passed through transparent frame containing a packed catalyst bed. The catalyst bed is comprised of photocatalyst particles (300-425 im) and particle support. The frame is irradiated with sunlight as a direct source of both UV light and visible light, to facilitate the reduction reaction as the reactant stream contacts the photocatalyst. The product stream can be separated away from the frame, and separated into individual streams using a suitable gas-liquid separator (not shown), methanol can be separated from liquid stream by distillation.

A recycle stream passes to the HPLC pump to once again capture carbon by carbon dioxide absorbance, to maximise CO₂ conversion.

The entire reaction system operates under ambient temperature and pressure.

The present invention shows various improved methods of converting carbon dioxide to methanol fuels using photocatalytic reduction and forming methanol and/or formic acid from carbon dioxide that has greater viability for scale up to a commercial scale.

As such, the present invention can increase capturing carbon dioxide (CO₂) from the atmosphere and utilising it to produce hydrocarbon fuels. Carbon capture and utilisation (CCU) can assist in overcoming the global energy crisis, providing energy security while combatting climate change simultaneously.

1. A method of forming methanol, comprising:
irradiating a mixture comprising water, carbon dioxide, carbon absorbent, and a photocatalyst, with visible light and UV light to reduce the carbon dioxide and form methanol,
wherein the photocatalyst comprises zinc oxide and manganese oxide.

2. The method of forming methanol as claimed in claim 1, carried out at ambient temperature, or at ambient pressure, or both ambient temperature and ambient pressure.

3. The method of forming methanol as claimed in claim 1, wherein the photocatalyst comprises zinc oxide and manganese oxide having an octahedral framework such as cryptomelane such as OMS-2.

4. The method of forming methanol as claimed in claim 1, wherein the carbon absorbent is at least one of an amine or oxide.

5. The method of forming methanol as claimed in claim 4, wherein the carbon absorbent is selected from the group comprising: triethylamine, dimethylformamide, calcium oxide, magnesium oxide and any mixture thereof.

6. The method of forming methanol as claimed in claim 1, wherein the volume ratio of the carbon absorbent to water is greater than 1:1.

7. The method of forming methanol as claimed in claim 6, wherein the volume ratio of the carbon absorbent to water is greater than 3:1.

8. The method of forming methanol as claimed in claim 1, wherein the carbon absorbent is a mixture of trimethylamine and dimethylformamide, and wherein the volume ratio of the carbon absorbent to water is 4:1.

9. The method of forming methanol as claimed in claim 1, wherein the weight ratio of zinc oxide to manganese oxide in the photocatalyst is in the range 50:1 to 1:50.

10. The method of forming methanol as claimed in claim 9, wherein the weight ratio of zinc oxide to manganese oxide in the photocatalyst is in the range 10:1 to 1:10.

11. The method of forming methanol as claimed in claim 10, wherein the weight ratio of zinc oxide to manganese oxide in the photocatalyst is in the range 3:1 to 5:1, such as 4:1.

12. The method of forming methanol as claimed in claim 1, wherein the UV light has a wavelength in the range of 350-400 nm.

13. The method of forming methanol as claimed in claim 1, wherein the visible light has a wavelength in the range of 600 nm to 800 nm.

14. The method of forming methanol as claimed in claim 1, wherein the irradiation is provided by direct sunlight.

15. The method of forming methanol as claimed in claim 1, further comprising the steps of:
passing the carbon dioxide through the water and carbon absorbent to provide a wholly or substantially saturated solution; and
passing the saturated solution across the photocatalyst.

16. The method of forming methanol as claimed in claim 1, further comprising the step of:
separating the methanol from the water and the carbon absorbent.

17. (canceled)

18. (canceled)

19. A method of forming formic acid, comprising:
irradiating a mixture comprising water, carbon dioxide, carbon absorbent, and a photocatalyst, with visible light and UV light to reduce the carbon dioxide and form formic acid,
wherein the photocatalyst comprises zinc oxide and manganese oxide,
further comprising one or more of:
the method is carried out at ambient temperature or ambient pressure, or both ambient temperature and ambient pressure;
the photocatalyst comprises zinc oxide and manganese oxide has an octahedral framework such as cryptomelane such as OMS-2;
the carbon absorbent is at least one of an amine or oxide;
the carbon absorbent is selected from the group comprising: triethylamine, dimethylformamide, calcium oxide, and any mixture thereof;
the volume ratio of the carbon absorbent to water is greater than 1:1;
the volume ratio of the carbon absorbent to water is greater than 3:1;
the carbon absorbent is a mixture of trimethylamine and dimethylformamide, and wherein the volume ratio of the carbon absorbent to water is 4:1;
the weight ratio of zinc oxide to manganese oxide in the photocatalyst is in the range 50:1 to 1:50;
the weight ratio of zinc oxide to manganese oxide in the photocatalyst is in the range 10:1 to 1:10;
the weight ratio of zinc oxide to manganese oxide in the photocatalyst is in the range 3:1 to 5:1;
wherein the UV light has a wavelength in the range of 350-400 nm;
the visible light has a wavelength in the range of 600 nm to 800 nm;
the irradiation is provided by direct sunlight;
the method further comprises the steps of passing the carbon dioxide through the water and carbon absorbent to provide a wholly or substantially saturated solution; and passing the saturated solution across the photocatalyst;
the method further comprises the step of separating the methanol from the water and the carbon absorbent; and/or
the method further comprises the step of recycling the water and the carbon absorbent for further carbon dioxide absorption.

20. (canceled)

21. Apparatus for forming methanol, formic acid, or a mixture of methanol and formic acid, comprising:
a mixing station for mixing water, carbon dioxide, and carbon absorbent,
a photocatalyst station configured to be irradiated with visible light and UV, wherein the photocatalyst comprises zinc oxide and manganese oxide.

22. The apparatus as claimed in claim 21, further comprising a gas/liquid separator after the photocatalyst station for separating the methanol, formic acid, or a mixture of methanol and formic acid, from the water and carbon absorbent.

23. The apparatus as claimed in claim 21, further comprising a recycling pathway to pass the water and carbon absorbent after the photocatalyst station and any gas/liquid separator, to the mixing station.

24. (canceled)

25. (canceled)

26. (canceled)

27. (canceled)

28. (canceled)
